(19) United States
(12) Patent Application Publication
(10) Pub. No.: US 2022/0280208 A1
(43) Pub. Date: Sep. 8, 2022

(54) MAGNETIC PROSTHETIC NAIL AND METHOD OF IMPLANTATION OF THE SAME

(71) Applicant: Ian Yu, Vancouver (CA)

(72) Inventor: Ian Yu, Vancouver (CA)

(21) Appl. No.: 17/190,438

(22) Filed: Mar. 3, 2021

(51) Int. Cl. A61B 17/72; B33Y 80/00

(52) U.S. Cl. CPC A61B 17/7291; A61B 17/7241; B33Y 80/00; A61B 2017/00876

(57) ABSTRACT

The magnetic implant system is provided to resolve the issue or deformed or removed nail from the fingers or toes using the magnetic implant and an artificial nail. The magnetic implant system of present invention is made of an implant made of a permanent magnet or rare earth metal coated with an inert metal to prevent infection at implanted location of the body, and an artificial nail made with 3D printing and digital scanning or otherwise, coated with the metallic paint at the backside to attract the magnetic field of the implant to hold its position at nail seat of finger or toe. According to one another embodiment, the implant is made of a metal while the backside of the artificial nail is made of a magnet.

### FIELD OF INVENTION

The present invention relates to the field of the method of implant of prosthetic nail. More particularly, it relates to an assembly of magnetic prosthetic nail and a magnetic implant for a finger or toe, and a method of implantation of the same over the finger or toe of the user.

### BACKGROUND OF INVENTION

Minor fingernail and toenail problems are common but there are incidents like repetitive trauma and/or infection which creates deformities in nails resulting in fungal infection, thickness, discoloration, or crumbly toenail or fingernails. Nail loss or deformity is not only unsightly in appearance but can also be dysfunctional. Oral therapies treating nail deformity are hazardous to the incapacitating liver and have less than 50% effectiveness and often lead to regrowth of the traumatized nails with root or nail matrix damage. Also, sometimes the body does not respond to these oral medications as well. Also, there are topical medication therapies which are helpful, but these therapies are expensive and demand a daily dose of therapy for an extensive period of time and efficacy is also low. However, these therapies are not helpful for nail matrix or root of the nail. Laser technology has been implemented and found more expensive and similarly frustrating in such cases. Apart from these therapies, removal of the nail to allow it to grow back often results in a recurrent deformity. Removal of the nail for good with a matrixectomy (i.e. the permanent removal of the nail) leaves a cosmetically unpleasant result.

There are numerous inventions which talks about using magnetic implant in the body to provide therapeutic benefits to a patient. For example, US 20080306324 discusses about methods and devices where one or more magnetic or magnetisable implant provides therapeutic benefits to a patient. The implant may be useful for expanding the range of motion of joints or dynamically providing different responses to changing conditions in the body where the implant is placed. An electromagnet is placed on or in a bone on one side of a joint, and another electromagnet or magnetically active material is placed on or in a bone on the opposing side of the joint. The electromagnet may be continuously energized to relieve pressure in the joint space, or may be energized in response to forces applied to the joint.

Moreover, there are also inventions which talk about creation and implantation of an artificial nail in the treatment of deformed or missing nails. US 20110226265 talks about the creation and implantation of an artificial nail in the treatment of deformed or missing nails includes preparing the nail bed. A polypropylene mesh is applied and anchored to the nail bed. KeryFlex is applied to the polypropylene mesh. However, this invention does not talk about using magnets to treat the deformity.

Apart from these two, there are inventions which talk about magnetic finger nails. GB 2575274 describes an artificial finger nail system which uses a first part which is an adhesive magnet. The first part adheres to the real finger nail. A second part is a decorative artificial nail and is also magnetic. The second part adheres to the first part via the magnets. The system provides a clean and relatively quick method of applying artificial finger nails.

Furthermore, US 20130006356 talks about a prosthetic implant devices and related methods. The prosthetic implant devices includes an internal component and an external component. The internal component has an implant portion associated with one or more rare earth magnets. The internal component being of a size and shape suitable for surgical implantation into the residual limb of the amputee. The implant portion being of a size and shape suitable for surgical implantation into a bone within the residual limb of the amputee. The one or more rare earth magnets generating at least one magnetic field. The external component having a prosthetic connection associated with a magnetic element. The magnet element being in adaptable magnetic association with the at least one magnetic field generated by the one or more rare earth magnets.

However, none of the above mentioned inventions talk about a coated magnetic implant designed to be secured into bone on the toe or fingers to create a magnetic field which attracts on magnetic artificial or prosthetic nail. Thus, to overcome the above mentioned problem, there is a need for an invention which provides a cost effective solution to nail deformity without taking medicines and also can be a used as a cosmetic replacement.

### SUMMARY

The present disclosure overcomes one or more shortcomings of the prior art and provide additional advantages discussed throughout the present disclosure. Additional features and advantages are realized through the techniques of the present disclosure. Other embodiments and aspects of the disclosure are described in detail herein and are considered a part of the claimed disclosure.

The subject matter disclosed by present invention is a system of magnetic implant and a magnetic nail and method of implantation of the same within the finger or toe, in place of deformed or removed natural nail. The present implant system is comprising of a magnetic implant designed to be secured into bones on the toes or fingers using surgical screws to permanently fix the magnetic implant there. According to one embodiment, the magnetic implant is coated with any other material to prevent corrosion and rejection when implanted. The magnetic implant is most preferably coated with any inert metal such as gold.

According to one embodiment, once the coated magnetic implant is implanted over the bone within the finger or the toe, using the screw or any other mean to secure the implant at said location, the magnetic implant creates a magnetic field around the finger or toe which attracts all the magnetized or metallic material. According to one embodiment, the present implant system includes a magnetic nail or an artificial/prosthetic nail made of metal or magnetized to attract with the magnetic field of the magnetic implant secured on the bone of the finger or the toe and stays at the location of the nail.

Thus, the present invention provides a magnetic implant assembly having an artificial magnetized nail made with digital scanning and 3D printing or otherwise, and a magnetic implant to provide user a new nail in place of any deformed or damaged natural nail. Normally, when the natural nail gets damaged or removed from the finger due to any reason, it feels and looks bad cosmetically. Sticking artificial nail using adhesive makes it painful at the time of removing or changing of said artificial nail. Hence, the present invention provides a complete magnetic implant system having a magnetic implant capable of being implanted and secured within the finger or toe, attached to the bone, and a magnetized or metallic artificial nail that attracts to the magnetic field of the implant and secures at the position of removed/damaged nail.

According to one embodiment, since the magnetized or metallic nail is not attached/fixed over the finger using any adhesive or permanent attachment member, replacing or changing said nail with other artificial magnetized or metallic nail becomes painless and more easier. According to one embodiment, the artificial nail is coated with metallic paint from one side only which may attract with the magnetic field and hold the place over the nail seat. Thus, the present implant system eliminates need of a prolonged use of an ineffective topical solutions and pills.

### BRIEF DESCRIPTION OF DRAWINGS

The foregoing summary, as well as the following detailed description of the invention is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows view of implant assembly of present invention with a magnetic implant secured over a finger bone.

FIG. 2 shows a present magnetic implant assembly connected over the toe.

### DETAILED DESCRIPTION OF INVENTION

The present invention overcomes the aforesaid drawbacks of the above, and other objects, features and advantages of the present invention will now be described in greater detail. Also, the following description includes various specific details and are to be regarded as merely exemplary. Accordingly, those of ordinary skill in the art will recognize that: without departing from the scope and spirit of the present disclosure and its various embodiments there may be any number of changes and modifications described herein.

The magnetic implant system for artificial finger or toe nail is provided to be secured magnetically over the nail seat, in place of natural nail, when the natural nail gets damaged or removed due to many reasons including repetitive trauma and/or infection. When the natural nail gets damaged or removed because of such reasons, it often results in thickened, discolored, crumbly and deformed nails. Further, the oral therapies are most of the time in-effective and hazardous to other body organs. Thus, according to one embodiment, the present invention provides a magnetic nail implant system, wherein the present system is made of inert coated magnetic implant to be implanted within the finger or toe and secured over the bone using any attachment mean, such as coated screws. Both the magnetic implant and connection means are coated with the inert metal to avoid infection and rejection within the body due to the implant.

According to one embodiment, the magnetic implant is made of a rare metal or any permanent magnet which can be of any possible geometric shape. For instance, the shape of the magnetic implant is made as a circular in shape but not limited and can be of any possible geometrical shape. According to one embodiment, the present artificial magnetic implant system includes a metal coated nail that attracts with the implanted magnetic implant and secures over the nail seat without displacing from there.

According to one embodiment, the magnetic implant system of present invention, wherein the artificial nail is made of any material that looks more realistic to the natural nail and coated with metallic color from only one side to attract with the implanted magnet to hold its position over the finger.

According to one more embodiment, the magnetic implant system is made of a metallic implant and an artificial nail having a magnetic backing. According to this embodiment, the implant of the system is made of any metal capable of attracting a magnet and also coated with an inert metal i.e. gold. While, the artificial nail includes a magnetic backing to attract the metal implanted within the finger or toe.

According to one embodiment, the person having any deformity in finger or toe nail may use the present magnetic implant system, wherein the coated implant piece is fixed within the finger or toe over the bone using a screw or any other connection mean and left it until the finger heals properly with the implant present within it. After that, the artificial nail is provided which attracts with the implant present within the finger (in both the abovementioned embodiments) to hold its place over the finger at nail seat.

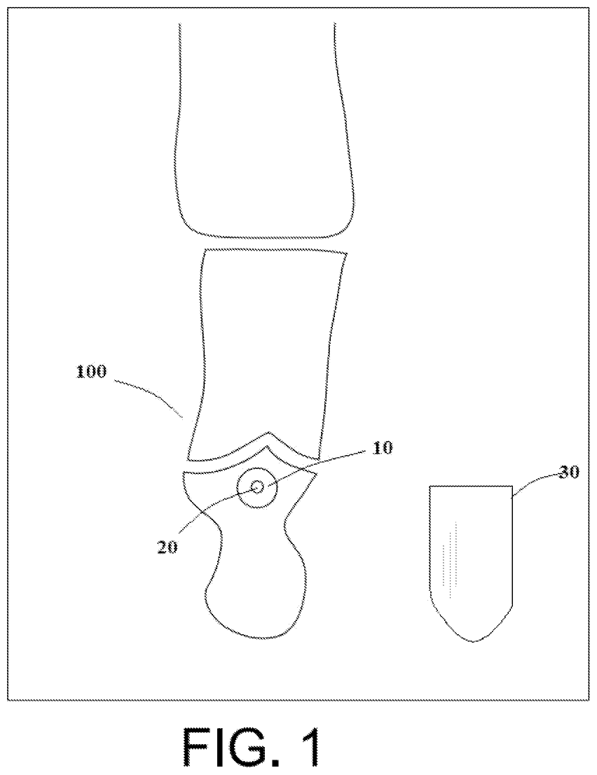

Referring to FIG. 1 now which discloses a magnetic implant assembly for a finger nail 100. According to present invention, the magnetic implant 10 is surgically fixed over the distal phalange bone of the finger using the screw 20. According to one embodiment, the present magnetic implant 10 may be attached over the finger bone using any other attachment means. According to present embodiment, both the magnetic implant 10 and the attachment means 20 are coated with an inert material such as gold so that the body may accept the implant 10 without any fear of infection.

According to present embodiment, the magnetic implant assembly of present invention further includes a metallic artificial finger nail 30 of present magnetic implant assembly which is made in the shape of a natural nail such that it can easily sit over the nail seat of the finger from where the natural nail has been removed due to any reason, while the magnetic field created by the magnetic implant 10 around the finger attracts the metallic artificial nail 30 and holds it at nail seat without need of any adhesive. According to one embodiment, the implant 20 of present magnetic implant assembly is made of any magnet attracting metal coated with an inert material, while the artificial nail 30 includes a layer of magnetic material at the bottom side of the nail 30 so that it can attract the metallic implant fixed within the finger to hold its place over the nail seat of the finger.

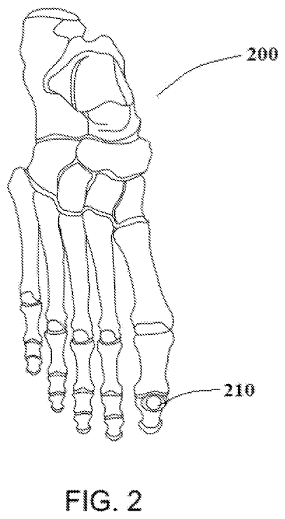

Now, FIG. 2 shows view of a foot 200 with a magnetic implant 210 fixed over the base of the distal phalange of the toe. According to present embodiment, the implant is fixed over the bone of the toe by drilling the bone for passing the screw through the bone to hold the implant over the bone. According to one embodiment, the assembly of present invention may include any other mean to permanently attach the implant over the bone. The implant with the attachment mean 210 is coated with an inert material so that the body may accept the implant.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A magnetic nail implant assembly for a finger or toe is comprising of:
a magnetic implant piece to get implanted within the finger or toe;
a connection mean to fix the magnetic implant piece over a phalange bone of the finger or toe; and
a metallic artificial nail to attract with a magnetic field of the implanted magnetic implant piece and sit over a nail seat of the finger without use of an adhesive.

2. The magnetic nail implant assembly of claim 1, wherein the magnetic implant piece is made of a permanent magnet.

3. The magnetic nail implant assembly of claim 1, wherein the connection mean is a screw.

4. The magnetic nail implant assembly of claim 1, wherein both the magnetic implant piece and the connection mean is coated with an inert material to reduce the risk of infection, corrosion and rejection.

5. The magnetic nail implant assembly of claim 1, wherein according to one embodiment, an implant piece is made metallic and an artificial nail made with 3D printing and digital scanning or otherwise is made with a magnetic bottom layer to attract the implant piece made of metal.

6. A method of implantation of a magnetic nail implant assembly is comprising steps of:
a. surgically making hole within a phalange bone of the finger or toe from where a natural nail have been removed;
b. placing a magnetic implant piece over the phalange bone;
c. fixing the magnetic implant piece with a connection mean over the phalange bone; and
d. placing an artificial metallic nail over a nail seat of the finger having the implant piece.

7. The method of implantation of claim 6, wherein the connection mean passes through the hole within the phalange bone.

8. The method of implantation of claim 6, wherein the connection mean is a screw.

9. The method of implantation of claim 6, wherein the magnetic implant piece and screw are coated with an inert material.

10. The method of implantation of claim 6, wherein the magnetic field of the magnetic implant piece attracts and holds the artificial metallic nail over the nail seat of the finger.

11. The method of scanning the nail bed and 3D printing an artificial mail or otherwise.
